(19) United States
(12) Patent Application Publication
(10) Pub. No.: US 2004/0171142 A1
(43) Pub. Date: Sep. 2, 2004

(54) Method for producing a plurality of identical copies of a two-dimensional test array of probe molecules

(76) Inventor: Ronald Frank, Braunschweig (DE)

Correspondence Address: Ronald R. Santucci, Frommer Lawrence & Haug, 745 Fifth Avenue, New York, NY, 10151, US

(21) Appl. No.: 10/473,847

(22) PCT Filed: Apr. 5, 2002
(86) PCT No.: PCT/EP02/03824

(30) Foreign Application Priority Data
Apr. 5, 2001 (DE) 101-17-135.8

(51) Int. Cl. C12M 1/34; G02B 6/255; C03B 37/15

(52) U.S. Cl. 435/287.2; 65/410

(57) ABSTRACT

The invention relates to a method for producing a plurality of identical copies of a two-dimensional closest-packed test array of probe molecules used to detect target biomolecules on the basis of fiber bundles that are cut to desired lengths.

The invention relates to a method for the production of a large number of identical copies of a planar test array of probe molecules for the detection of target molecules, on the basis of fibre bundles which are cut at desired intervals.

Collections of large numbers of different probes, probe molecules or test compounds that are deposited/bound/immobilised in an organised manner on a plane surface are referred to in scientific usage as arrays. Such arrays permit rapid simultaneous testing/assaying of all the probes/probe molecules/compounds by analysis of their interaction with an analyte or with a mixture of analytes, for example in biological samples, that is to say target bio-molecules. The advantage of an array over simultaneous testing/assaying using immobilised probes, probe molecules or test compounds on mobile elements, such as, for example, on beads, is that, in an array, the type (chemical structure and/or identity) of immobilised probe, probe molecule or test molecule is precisely known by the position in the array surface and a local test signal (this can be produced as a result of interaction with the target molecule, for example by binding, or can disappear, for example as a result of enzymatic conversion of the probe by the target molecule, and can thus serve indirectly for detection) can therefore be immediately assigned to a type of molecule. In miniaturised form especially, arrays having biological probes, probe molecules or test molecules are also called biochips.

Important examples of such arrays are:

nucleic acid arrays of DNA fragments, cDNAs, RNAs, PCR products, plasmids, bacterio-phages, synthetic oligonucleotides or also synthetic PNA oligomers which are read by means of hybridisation (formation of a double-strand molecule) to complementary nucleic acid analytes;

protein arrays of antibodies, proteins expressed in cells, phage fusion proteins (“phage display”) and

compound arrays of synthetic peptides, analogues thereof, such as peptoids, oligo-carbamates etc., or, in general, organic chemical compounds which are read, for example, by means of binding to affine protein analytes or other analytes or, for example, by means of enzymatic conversion.

Such arrays and the methods and apparatus developed for them are used in fundamental biological research and especially also in medical diagnostics and the development of pharmaceuticals. Other areas of research in the natural sciences, such as, for example, catalyst development and material sciences, are beginning to adopt such concepts successfully. A pre-requisite for advantageous routine use of such arrays is inexpensive, rapid and fully automated production thereof with a high density and diversity of test structures (information content).

Such arrays are currently produced according to two different principles by depositing the probes, probe molecules or test molecules on already prepared material surfaces (an up-to-date summary is given by S. Wölfl in: transcript Laborwelt 2000, 3, 12-20):

a) by single distribution of solutions of prefabricated probes, probe molecules or test compounds over the surface,

b) by repeated, serial distribution of solutions of building blocks for the chemical synthesis of the probes, probe molecules or test compounds in situ over the surface.

Previously known chip configurations use either a rectangular x/y arrangement of the array elements, which are produced by metered addition by means of corresponding x/y pipetting stations or by means of photolithographic or printing masks manufactured accordingly, or a circular rφ arrangement, which is produced by rotational movement of the chip surface (rφ arrays) and a metering device timed for rapid operation. It is thereby possible to obtain densities of up to 1 million probes, probe molecules or test compounds per cm³ or of a few square micrometres per individual area.

For use in routine medical diagnostics there are very strict requirements, however, as to the reproducibility of the analysis results over very large numbers (several millions) of tests. This demands an almost identical quality of the chips (arrays) of one production batch and also of different production batches. All of the production methods mentioned above, however, have a major fundamental disadvantage which is that each array so produced is only to a limited extent comparable to a second “identically” produced array, since each array element is produced in a single process.

This is also true when the same solution of a probe, probe molecule or compound is distributed over the same locations of a series of different arrays. Errors or deviations occur as result of metering inaccuracies, inhomogeneity of the surface characteristics and functionality and variable reaction yields of the immobilisation or synthesis steps.

Such error variations become the greater the smaller are the spatial dimensions of the array elements and become exponentially greater with the number of steps needed for the production of each individual array element.

Owing to the differing chemical structure and properties of the various probes, probe molecules or test molecules in the array elements, reference elements also are only relatively capable of correcting such variabilities. Quality testing of each individual array is not an option, since this is too expensive and many tests cannot be carried out reversibly, that is to say the array would be irreversibly altered by the quality control.

Fislage and Teterin have described in DE 198 03 077 C1 a “method for the production of structured test bodies for the specific detection of individual reactants of receptor substance-ligand substance complexes”, in which layers of materials to which the reactant substances are bound are stacked and adhesively bonded together to form a three-dimensional body and that three-dimensional body is subsequently cut at an angle to the plane of the layers into thin layers again using a microtome. The layers so produced are composed of thin strips lying adjacent to one another, each strip containing a different reactant substance so that, in a biological test, several different reactant substances can be simultaneously investigated in parallel in one test body. They have therefore proposed a process for cutting microtome-fine thin slices from a body composed of segments charged with different substances. The strip-like test bodies produced by that process use, however, only one dimension for arranging a large number of reactant substances for a parallel test. These reactant substances described in DE 198 03 077 can also be understood as probe molecules.

Anderson, Anderson and Braatz (WO 01/09607 A1) have overcome that disadvantage by making the three-dimensional body that is to be cut up from thread segments which are firstly charged with the various reactant substances and then placed side-by-side in parallel in a rectangular grid arrangement. That production process can certainly be used for relatively thick fibres, such as those described in the Examples. If, however, the thickness of the fibres is in the micrometre range and several hundred thousand of them are to be arranged in parallel, it would be necessary to design high-precision machinery.

The object of the invention is therefore to overcome the disadvantages or problems associated with the above-mentioned prior art.

The invention accordingly relates to a method for the production of a large number of identical copies of a planar test array of probe molecules for the detection of target molecules, in which

(a) a large number of fibres is used as the starting point, wherein each fibre has only one type of probe molecule for the detection of one type of target molecule and the number of fibres corresponds to at least the number of different types of target molecules to be detected,

(b) the large number of fibres is arranged in parallel orientation,

(c) the fibres so arranged are bundled to form a fibre bundle and are brought into the closest packing (viewed at right angles to the cross-section of the fibres),

(d) the arrangement of the individual fibres with respect to one another in the fibre bundle is unalterably fixed so that each fibre occupies a geometrically defined position and a solidified fibre bundle is obtained, and

(e) the solidified fibre bundle is cut at desired intervals at right angles to the length of the fibres, producing a large number of identical copies of a planar test array having a pattern of probe molecules for the detection of target molecules in geometrically defined positions on the cut surfaces.

According to the invention, the prior art of WO 01/09607 is improved in the respect that the process of placing the fibres against one another is made into a simple, self-organising process. The fibres are first arranged approximately parallel to one another while spaced apart and can thus be placed in the desired order or charged with the probe molecules layer-by-layer only during their arrangement. The arranging process does not require high precision, but the correct arrangement must merely be ensured.

The subsidiary claims relate to other advantageous and/or preferred embodiments of the invention.

According to one embodiment, the invention relates to a method in which a large number of probe molecules is immobilised on the respective surfaces of a large number of fibres or is synthesised on those surfaces.

According to a further embodiment, the invention relates to a method in which a large number of fibres is produced by extrusion of a large number of basic fibre materials each having a large number of probe molecules immobilised thereon.

According to one embodiment of the invention, the fibres may consist, for example, of porous synthetic material, especially polyester, polyurethane or nylon, cellulose, cellulose acetate, cotton or silk. The fibres do not necessarily have to be porous, however. It would also be possible to use fibres consisting, for example, of glass, metal, metal oxides or semimetal oxides. In the detection process, however, only a signal in the region of the “coating” on the fibre would form, that is to say a kind of annular signal around the fibre, which would not, of course, be as intense as a signal extending over the entire cross-section of the fibre.

If basic fibre materials having probe molecules immobilised thereon are to be used for extrusion, the person skilled in the art can be guided by the prior art that is relevant to gentle extrusion of functionalised basic fibre materials; cf., for example, Science, 295 (2002) 472 and Schnegelsberg, Handbuch der Faser, Theorie und Systematik der Faser, 1999, ISBN 3 871 506 249.

According to a further embodiment of the invention, combination of the fibres to form a fibre bundle is effected, for example, by twisting or intertwining in the same direction.

Twisting of the bundle automatically brings the fibres as close as possible to one another. This produces a somewhat different arrangement of the fibres by the closest packing principle, but the spatial position relative to one another is not thereby changed. A further advantage is the greater compactness of the test array.

According to a further embodiment of the invention, unalterable fixing of the arrangement of fibres with respect to one another and the solidification of the fibre bundle are effected, for example, by embedding or polymerisation into a solidifiable material and subsequent hardening or full polymerisation or freezing thereof.

According to a further embodiment of the invention, the solidifiable material is, for example, paraffin, gelatin, polyacrylamide, epoxy resin, polyethylene glycol (PEG), an aqueous polyvinyl alcohol solution or an aqueous polyvinyl alcohol/PEG solution. In principle, any material suitable for microtomy or cryotomy is suitable. The person skilled in the art will be familiar with matching the hardnesses of solidifiable material and fibre.

According to a further embodiment of the invention, the solidified fibre bundle is cut, for example, with a microtome, for example with an ultra-microtome, or a cryotome; the cuts can be made perpendicular to or at an angle to the axis of the fibre bundle.

The invention further relates to a planar test array of probe molelcules for the detection of target molecules, the test array being formed by the most compact 2-dimensional arrangement or closest packing of planar elements having a surface area, and specifically equal surface areas, in one and the same plane, wherein each element has only one type of probe molecule for the detection of one type of target molecule and wherein the number of fibres corresponds to at least the number of different types of target molecules to be detected.

The invention further relates to a planar test array of probe molecules for the detection of target molecules, obtainable by a method according to the invention.

In the planar test array according to the invention, the target molecules may be target biomolecules.

In the planar test array according to the invention, the elements having equal surface areas may have a disc shape, especially a circular disc shape, an elliptical disc shape or a hexagonal disc shape, and may be present in one and the same plane in the closest packing, especially in the hexagonally closest packing (viewed at right angles to the plane of the disc).

The invention further relates to a medical or diagnostic apparatus comprising one or more, identical or different planar test array(s) of probe molecules for the detection of target molecules according to the invention.

The invention further relates to a kit comprising a number of identical or different planar test arrays of probe molecules for the detection of target molecules according to the invention.

The invention further relates to a kit comprising one or more, identical or different planar test array(s) of probe molecules for the detection of target molecules according to the invention and one or more reagents for the detection of target molecules that bind to probe molecules.

The invention also provides the use of a planar test array according to the invention, of a medical or diagnostic apparatus according to the invention or of a kit according to the invention for the detection of target molecules.

The probe molecules used according to the invention may, for example, each be a partner of a specifically interacting system of complementary binding partners.

As a result of the binding of the complementary binding partners, a detectable signal can be produced, for example directly as a result of the binding per se, or indirectly because a further marked molecule binds specifically to the probe/target biomolecule conjugate (sandwich assay). A signal present before binding may also disappear, for example because a marker attached to a probe is removed or is deactivated in another way (when, for example, the target biomolecule is an enzyme).

The specifically interacting system of complementary binding partners may, for example, rely on the interaction of nucleic acid/complementary nucleic acid, peptide nucleic acid/nucleic acid, enzyme/substrate, receptor/effector, lectin/sugar, antibody/antigen, avidin/biotin, streptavidin/biotin.

The above-mentioned antibodies may, for example, be polyclonal, monoclonal, chimaeric or single-chain antibodies or a functional fragment or derivative of such an antibody.

The invention is described in more detail below, without any limitation, with the aid of illustrative embodiments and with reference to the drawings.

In the drawings:

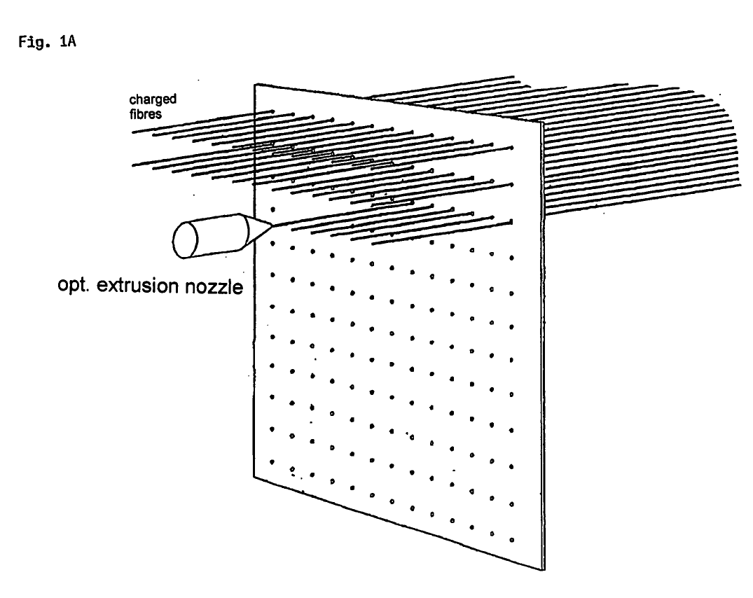

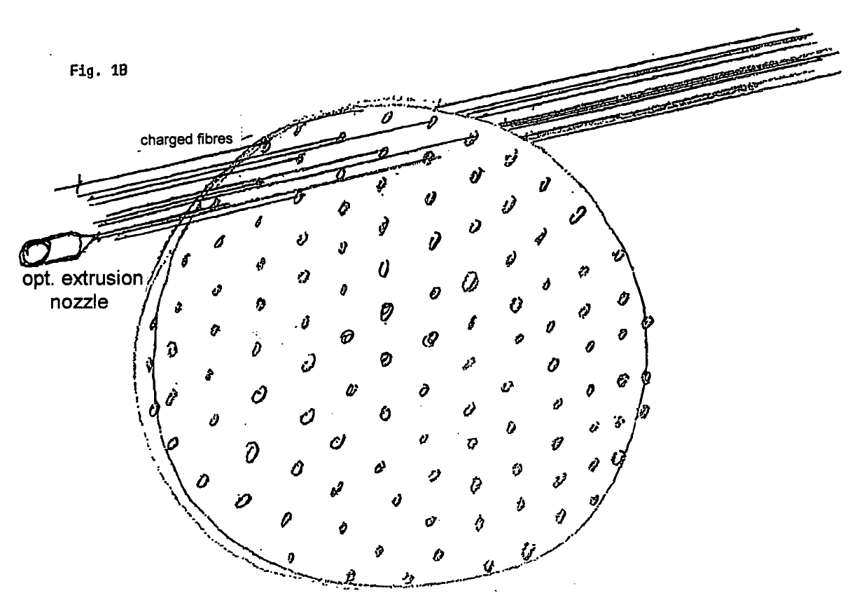

FIG. 1A is a diagrammatic view of individual steps of an embodiment of the method according to the invention and also an embodiment of a product obtained by the method according to the invention;

FIG. 1B is a further diagrammatic view of individual steps of an embodiment of the method according to the invention;

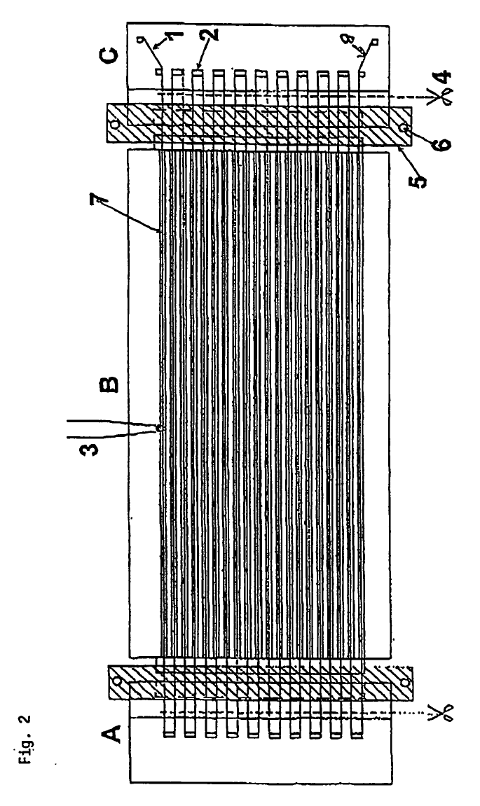

FIGS. 2 and 2A to 2C are further diagrammatic views of individual steps of an embodiment of the invention;

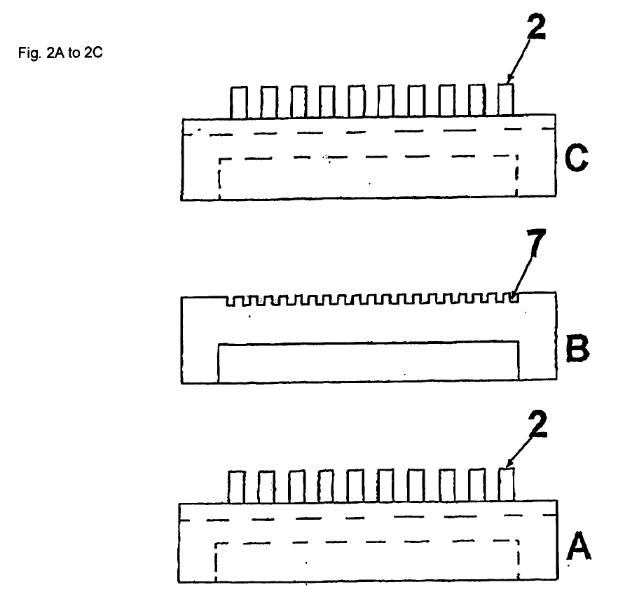

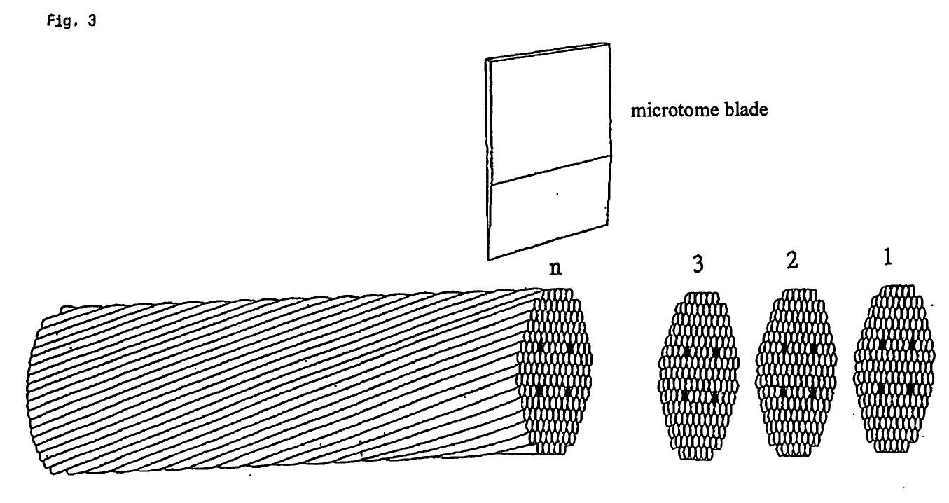

FIG. 3 shows a fibre bundle compacted according the invention and test arrays obtained by means of the fibre bundle; and

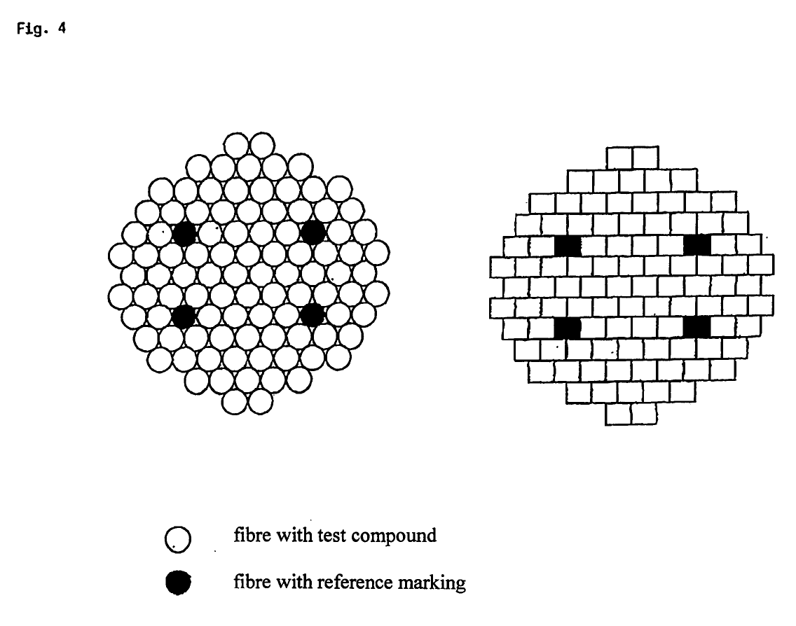

FIG. 4 shows front views of fibre bundles compacted according to the invention or of test arrays according to the invention.

As shown in FIG. 1, a basic fibre material having a large number of probe molecules immobilised thereon can be used as the starting point, and the material can be spun by means of nozzles of a bundle or a battery of nozzles into fibres approximately parallel to one another which are fixed to a plate or a perforated plate arranged opposite the nozzles. For the sake of simplicity, only one nozzle is shown. Alternatively, already spun threads can be threaded through a perforated plate.

Whereas the plate shown in FIG. 1A is rectangular or square and is provided with holes arranged in a square grid pattern, the plate shown in FIG. 1B is approximately circular and the arrangement of holes is approximately hexagonal.

FIG. 2 is a plan view of a device for the parallel arrangement of fibres and FIGS. 2A to 2C are views in section. The device is provided with guide pins 2 which serve to lay out a continuous thread in meandering manner (beginning at 1 and ending at 8). As shown in FIG. 1B, the approximately rectangular base of the device is provided with channel-like grooves 7, channels or troughs which receive the individual parallel sections of the thread. In those grooves 7, the fibres can be impregnated or provided with a functionalisation. Plastics strips 5 serve as a support for and/or for covering the end portions of the parallel sections and can be welded and/or adhesively bonded to the end portions. A large number of pairs of strips 5, with their threads welded or adhesively bonded to them in meandering manner, can be stacked one on top of another with the aid of spikes which are guided through guide holes 6 provided in the strips 5.

FIG. 3 shows a twisted fibre bundle from which test arrays according to the invention are cut by means of a microtome blade. These test arrays are provided with four marks so that they can be oriented in analogous manner.

FIG. 4 shows test arrays having differing cross-sections of the fibres that are combined with one another.

According to the invention, the following method is proposed especially for the production of arrays for routine medical diagnostics, in which each array element of a large series of identical arrays is produced from an identical material:

Firstly, for example, the probe or probe molecules are immobilised on “one-dimensional” thread, wire or rod elements (“1D elements”). This can be done, for example, by binding appropriate probe molecules directly or via suitable linkers to reactive groups on the surface of the “1D elements”. For example, a solution of the probe molecules can be allowed to run down a thread fastened vertically, or the thread is drawn through a bath of a solution of the probe molecules or is placed in the bath. There are no particular limitations as to the method of application. Preferably, the thread is saturated/impregnated, for example, with a solution of the probe molecules, which, in the case of porous fibres, for example, such as cellulose or cotton threads, occurs automatically owing to the wick effect (absorption) and produces a homogeneous distribution of the solution of the probe molecules in the fibre in question.

The “one-dimensional” thread, wire or rod elements (“1D elements”) can then be arranged parallel lengthwise and then, for example in the same way as in the production of a rope, firmly combined with one another with the optimum close packing (e.g. intertwined) to form a “three-dimensional” array body (“3D body”). That can be achieved most simply, for example, by twisting the fibre arrangement in the same direction or by another kind of intertwining method. That 3D body is then impregnated with a solidifying material, which is not per se subject to any particular limitations. A cut is then made perpendicular to or at an angle to the axis of the combined “one-dimensional” array elements at one end of the 3D body, which can be done by any desired method. The cut surface then corresponds to a two-dimensional arrangement of array elements as in a conventional array (2D array). A large number of thin slices can then be cut off one after another and each slice will consist of an identical 2D array. These arrays can then be applied to a stable support and can be subjected to further treatment in the same manner as other, conventional arrays.

This new production method has the following advantageous features:

a) The “1D elements” can consist of prefabricated materials (rods, wires or threads) which are each charged with one probe, probe molecule or compound in a preceding complete process. Charging is like a process with which probes, probe molecules or compounds are immobilised on a surface or chemically synthesised there in situ in accordance with solid-phase synthesis principles. Simple examples are cellulose, cellulose acetate or cotton threads to the hydroxyl functions of which the compounds are chemically linked. A similar procedure can be applied to silk threads or synthetic material threads, especially threads based on polyesters, polyurethane or nylon. A great many examples of the immobilisation of molecules on surfaces that are suitable for combinations of probe/target biomolecules or, in general, for bioconjugation systems are to be found in the book “Bioconjugate Techniques” by G. T. Hermanson, Academic Press, 1996. A great many examples of systems for solid-phase synthesis are to be found in the book “Organic Synthesis on Solid Phase” by F. Z. Dörwaid, Wiley-VCH, 2000.

Alternatively, the “1D elements” can also be produced directly from, for example, a solution of a suitable basic material, in this case the probes, probe molecules or test compounds being already bonded covalently, ionically or mechanically to molecules of the basic material; cf., for example, WO 99/54 729. Production can be carried out, for example, by an extrusion process as in the production of synthetic fibres or of viscose, cellulose acetate or silk fibres. In this case, a rectangular, hexagonal or other shape of the fibres/wires can also be obtained by means of corresponding apertures of the extrusion nozzles.

That procedure ensures that each array element which is later obtained as a very small part will consist of the same material manufactured in large quantities in the preceding production process.

b) In one embodiment of the invention, the arrangement and combination of the “1D elements” can be compared to a rope-making process. The ends of the fibres are ordered in the arrangement for the desired array while spaced apart from one another and the threads are then twisted slightly. Twisting results in the bundle being held together firmly and compacted. That compaction is self-organising and reproducible. For exact determination of the final orientation of a 2D array section, reference fibres having, for example, a coloured, fluorescent or other suitable marking can be incorporated. Only one sorting operation is required for all the arrays of a series.

c) The cutting operation corresponds to conventional microtome technology with which extremely thin sections of biological material embedded in a suitable medium can be produced. Ultra-microtomes produce sections of as little as only 0.1 micrometre thickness. 10 million array slices of 0.1 μm thickness could therefore be produced from a 1 metre long 3D body without any difficulty.

The 3D body is for that purpose impregnated with a material suitable for microtomy (e.g. paraffin, gelatin, polyacrylamide, epoxy resin, polyethylene glycol (PEG), aqueous polyvinyl alcohol solution or aqueous polyvinyl alcohol/PEG solution) and the fibres are thus embedded or polymerised or frozen therein as the biological material would normally be. There are numerous directions on this subject by manufacturers of microtomes and ultra-microtomes that are suitable according to the invention.

d) The microtome sections can be placed on a stable support, such as glass, bonded and then further treated as desired, as specified in the instructions on the use of conventional arrays. The sizes of the array supports can be adapted to those of conventional arrays (e.g. microscope slides measuring 2.5×7.5 cm) so that commercial apparatus for treating and reading arrays can also be used.

It is possible both for one section to be arranged on one support and for an arrangement of several sections to be arranged on a common support (“array of arrays”). In the case of the latter arrangement, multiple identical arrays or also different arrays can be arranged. Between the sections of such “arrays of arrays”, small webs can be placed or produced, so that separate chambers are obtained and each individual section can be investigated with a different biological sample simultaneously.

The entire process can easily be fully automated.

### List of reference numerals

A right end piece for guiding fibre

B central piece for impregnation channels

C left end piece for guiding fibre

1 thread (beginning)

2 guide pin

3 pipette for filling impregnation channels 7

4 line for cutting the fibre plane

5 plastics strips for welding the fibre ends

6 guide hole for stacking fibre planes

7 channel for impregnating solution

8 thread (end)

1. A method for the production of a large number of identical copies of a planar test array of probe molecules for the detection of target molecules, in which
(a) a large number of fibres is used as the starting point, wherein each fibre has only one type of probe molecule for the detection of one type of target molecule and the number of fibres corresponds to at least the number of different types of target molecules to be detected,
(b) the large number of fibres is arranged in parallel orientation,
(c) the individual fibres are bundled to form a fibre bundle and are brought into the closest packing (viewed at right angles to the cross-section of the fibres),
(d) the arrangement of the individual fibres with respect to one another in the fibre bundle is unalterably fixed so that each fibre occupies a geometrically defined position and a solidified fibre bundle is obtained, and
(e) the solidified fibre bundle is cut at desired intervals at right angles to the length of the fibres, producing a large number of identical copies of a planar test array having a pattern of probe molecules for the detection of target molecules in geometrically defined positions on the cut surfaces.

2. A method according to claim 1, in which the individual fibres are bundled to form a fibre bundle (sub-fibre-bundle) and a number of such bundles is bundled to form a combined bundle (super-bundle).

3. A method according to claim 1 and/or 2, wherein, in steps (a) and (b)
(i) the large number of fibres is spun from a bundle of nozzles or
(ii) one continuous fibre is arranged in meandering manner in several planes and the large number of fibres can be formed by the parallel portions of the meanders or
(iii) a number of continuous fibres is arranged in meandering manner each in one plane of parallel planes and the large number of fibres can be formed by the parallel portions of the meanders.

4. A method according to at least one of the preceding claims, wherein a large number of probe molecules is in each case immobilised on the respective surfaces of a large number of fibres or is synthesised on those surfaces, wherein each surface can be formed by the accessible inner and outer surface.

5. A method according to at least one of claims 1 to 3, wherein a large number of fibres is produced by extrusion of a large number of basic fibre materials each having a large number of probe molecules immobilised thereon.

6. A method according to at least one of the preceding claims, wherein the fibres consist of porous synthetic material, especially polyester, polyurethane or nylon, cellulose, cellulose acetate, cotton or silk, especially natural silk.

7. A method according to at least one of the preceding claims, wherein the combination of the fibres to form a fibre bundle is effected by twisting or intertwining in the same direction.

8. A method according to at least one of the preceding claims, wherein the unalterable fixing of the arrangement of the fibres with respect to one another and the solidification of the fibre bundle are effected by embedding or polymerisation into a solidifiable material and subsequent hardening, full polymerisation or freezing thereof.

9. A method according to claim 7, wherein the solidifiable material is paraffin, gelatin, polyacrylamide, epoxy resin, polyethylene glycol (PEG), an aqueous polyvinyl alcohol solution or polyvinyl alcohol/PEG solution.

10. A method according to at least one of the preceding claims, wherein the solidified fibre bundle is cut with a microtome or a cryotome.

11. A planar test array of probe molecules for the detection of target molecules, wherein the test array is formed by the most compact 2-dimensional arrangement or the closest packing of planar elements having a surface area, and specifically equal surface areas, in one and the same plane.

12. A planar test array of probe molecules for the detection of target molecules, especially according to claim 11, wherein each element has only one type of probe molecule for the detection of one type of target molecule and wherein the number of elements corresponds to at least the number of different types of target molecules to be detected.

13. A planar test array according to at least one of claims 11 and 12, wherein the elements having equal surface areas have a disc shape, especially a circular disc shape, an elliptical disc shape or a hexagonal disc shape, and are present in the closest packing in one and the same plane, especially in the hexagonally closest packing (viewed at right angles to the plane of the disc).

14. A planar test array of probe molecules for the detection of target molecules, obtainable by a method according to at least one of claims 1 to 10.

15. A planar test array according to at least one of claims 10 to 14, in which the target molecules are target biomolecules, especially cells, viruses, phages, nucleic acids, peptide nucleic acids, proteins, enzymes, receptors, lectins, sugars, antibodies, antigens, avidin, streptavidin or biotin.

16. A medical or diagnostic apparatus comprising one or more, identical or different planar test array(s) of probe molecules for the detection of target molecules according to at least one of claims 10 to 15.

17. An apparatus according to claim 17, namely holding, washing or incubation apparatus for test array(s).

18. A kit comprising a number of identical or different planar test arrays of probe molecules for the detection of target molecules according to at least one of claims 10 to 15.

19. A kit comprising one or more, identical or different planar test arrays of probe molecules for the detection of target molecules according to at least one of claims 10 to 15 and one or more reagents for the detection of target molecules that bind to probe molecules.

20. The use of a planar test array according to at least one of claims 10 to 15 or of a medical or diagnostic apparatus according to claim 16 or 17 or of a kit according to one of claims 18 and 19 for the detection of target molecules, especially target biomolecules.
